(19) United States
(12) Patent Application Publication
(10) Pub. No.: US 2019/0338352 A1
(43) Pub. Date: Nov. 7, 2019

(54) ACCURACY OF BASE CALLS IN NUCLEIC ACID SEQUENCING METHODS

(71) Applicant: Omniome, Inc., San Diego, CA (US)

(72) Inventors: Alex NEMIROSKI, San Diego, CA (US); Sean STROMBERG, Encinitas, CA (US); John VIECELI, Encinitas, CA (US)

(73) Assignee: Omniome, Inc., San Diego, CA (US)

(21) Appl. No.: 16/361,034

(22) Filed: Mar. 21, 2019

Related U.S. Application Data
(60) Provisional application No. 62/659,897, filed on Apr. 19, 2018.

(51) Int. Cl. C12Q 1/6874; G16B 30/00

(52) U.S. Cl. CPC C12Q 1/6874; G16B 30/00

(57) ABSTRACT

A method of determining nucleic acid sequences can include steps of (a) obtaining signal data from a nucleic acid sequencing procedure carried out on an array of nucleic acid features; (b) extracting signals from each nucleic acid feature to produce multiple extracted signal traces that each correlate signal characteristics with sequencing cycle for a particular nucleotide type at a particular nucleic acid feature; (c) comparing the series of signals for different nucleotide types at each of the features to distinguish a candidate base call from background signals for each cycle at each feature; (d) applying a baseline adjustment to each series of signals based on the extracted background signals; and (e) comparing the adjusted signal traces for different nucleotide types at each of the features, thereby distinguishing adjusted signals having characteristics of a base call from adjusted background signals for each cycle at each feature.

### CROSS-REFERENCE TO RELATED APPLICATION

This application is based on, and claims the benefit of, U.S. Provisional Application No. 62/659,897, filed Apr. 19, 2018, which is incorporated herein by reference.

### BACKGROUND

The present disclosure relates generally to cyclical reactions carried out in multiplex formats and has specific applicability to sequencing nucleic acids in array-based platforms.

A variety of nucleic acid sequencing platforms are based on detection of fluorescently labeled components. Generally, genomic DNA fragments are arrayed as individual DNA colonies on a solid-support, the array is subjected to a chemical procedure that labels each colony according to the type of nucleotide that is present at a particular position in the genomic fragment, the labeled colonies are imaged, and the procedure is repeated. The sequence of nucleotides for each DNA fragment is determined from the series of labels observed at each DNA colony across the images.

Images acquired in sequencing procedures are prone to noise and interference from a variety of sources. Sequencing technologies generally include image corrections designed to correct known sources of noise and interference, such as optical crosstalk or phasing noise. These corrections assume a model for the source of noise and then determine the coefficients for that model. Take, for example, phasing correction. Phasing refers to the pernicious phenomena whereby a subset of genomic fragments falls behind or jump ahead of other fragments in the colony during the sequencing procedure. Over time, the increase in out of phase fragments leads to an overwhelming increase in noise. In the case of phasing correction, coefficients are determined to multiply the previous cycle signal intensities and the next cycle signal intensities to correct the current cycle signal intensities. However, due to assumptions regarding causation of the noise and due to the broad stroke attempt to correct all features using a single model, such corrections are often inadequate especially for longer more complex sequencing protocols that suffer from noise and interference of unknown origin.

Thus, there exists a need for improved image analysis and noise correction procedures. The present invention satisfies this need and provides related advantages as well.

### BRIEF SUMMARY

The present disclosure provides a method of determining nucleic acid sequences. The method can include steps of (a) obtaining signal data from a nucleic acid sequencing procedure carried out on an array of nucleic acid features; (b) extracting signals from each nucleic acid feature to produce multiple extracted signal traces, wherein each extracted signal trace correlates signal characteristics with sequencing cycle for a particular nucleotide type at a particular nucleic acid feature; (c) comparing the extracted signal traces for different nucleotide types at each of the features, thereby distinguishing an extracted signal having a characteristic of a candidate base call from extracted background signals for each cycle at each feature; (d) applying a baseline adjustment to each extracted signal trace based on the extracted background signals, thereby obtaining a adjusted signal trace for each nucleotide at each feature; and (e) comparing the adjusted signal traces for different nucleotide types at each of the features, thereby distinguishing adjusted signals having characteristics of a base call from adjusted background signals for each cycle at each feature, whereby nucleic acid sequences are determined from the sequence of the base calls at each of the features.

Also provided is an iterative method that includes the steps of: (a) obtaining signal data from a nucleic acid sequencing procedure carried out on an array of nucleic acid features; (b) extracting signals from each nucleic acid feature to produce multiple extracted signal traces, wherein each extracted signal trace correlates signal characteristics with sequencing cycle for a particular nucleotide type at a particular nucleic acid feature; (c) comparing the extracted signal traces for different nucleotide types at each of the features, thereby distinguishing an extracted signal having a characteristic of a candidate base call from extracted background signals for each cycle at each feature; (d)(i) applying a baseline adjustment to each extracted signal trace based on the extracted background signals, thereby obtaining a adjusted signal trace for each nucleotide at each feature, (d)(ii) comparing the adjusted signal traces for different nucleotide types at each of the features, thereby distinguishing an adjusted signal having a characteristic of a candidate base call from adjusted background signals for each cycle at each feature, (d)(iii) applying a baseline adjustment to each adjusted signal trace based on the adjusted background signals, thereby obtaining a series of iteratively adjusted signals for each nucleotide at each feature; and (e) comparing the adjusted signal traces for different nucleotide types at each of the features, thereby distinguishing adjusted signals having characteristics of a base call from adjusted background signals for each cycle at each feature, whereby nucleic acid sequences are determined from the sequence of the base calls at each of the features.

In some embodiments, the method can include steps of (a) obtaining luminescence image data from a nucleic acid sequencing procedure carried out on an array of nucleic acid features; (b) extracting luminescence signals from each nucleic acid feature to produce multiple series of luminescence signals, wherein each series of luminescence signals correlates luminescence intensity with sequencing cycle for a particular nucleotide type at a particular nucleic acid feature; (c) comparing the series of luminescence signals for different nucleotide types at each of the features, thereby distinguishing a candidate base as having the highest luminescence intensity from background luminescence signals for each cycle at each feature; (d) applying a baseline adjustment to each series of luminescence signals based on the extracted background signals, thereby obtaining a series of adjusted luminescence signals for each nucleotide at each feature; and (e) comparing the series of adjusted luminescence signals for different nucleotide types at each of the features, thereby distinguishing adjusted luminescence signals having characteristics of a base call from adjusted background luminescence signals for each cycle at each feature, whereby nucleic acid sequences are determined from the sequence of the base calls at each of the features.

An iterative version of the image-based method can include steps of (a) obtaining luminescence image data from a nucleic acid sequencing procedure carried out on an array of nucleic acid features; (b) extracting luminescence signals from each nucleic acid feature to produce multiple series of luminescence signals, wherein each series of luminescence signals correlates luminescence intensity with sequencing cycle for a particular nucleotide type at a particular nucleic acid feature; (c) comparing the series of luminescence signals for different nucleotide types at each of the features, thereby distinguishing a candidate base as having the highest luminescence intensity from background luminescence signals for each cycle at each feature; (d) (i) applying a baseline adjustment to each series of luminescence signals based on the background luminescence signals, thereby obtaining a series of adjusted luminescence signals for each nucleotide at each feature, (d)(ii) comparing the series of adjusted luminescence signals for different nucleotide types at each of the features, thereby distinguishing an adjusted luminescence signal having the highest luminescence intensity as a candidate base call from background luminescence signals for each cycle at each feature, (d)(iii) applying a baseline adjustment to each series of luminescence signals based on the adjusted background signals, thereby obtaining a series of iteratively adjusted luminescence signals for each nucleotide at each feature; and (e) comparing the series of adjusted luminescence signals for different nucleotide types at each of the features, thereby distinguishing adjusted luminescence signals having characteristics of a base call from adjusted background luminescence signals for each cycle at each feature, whereby nucleic acid sequences are determined from the sequence of the base calls at each of the features.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the operations set forth in the above methods.

### BRIEF DESCRIPTION OF THE DRAWINGS

FIG. 1 shows a diagram of an algorithm for correcting signal traces that have been separately extracted for individual nucleotide types and from individual clusters.

FIG. 2A shows a plot of signal intensity vs. cycle for A, C, T and G signals that have been extracted from a single nucleic acid cluster having been subjected to a Sequencing By Binding™ procedure.

FIG. 2B shows a plot of adjusted signal intensities vs. cycle for the A, C, T and G signals after a single iteration of baseline correction for the data shown in FIG. 2A.

FIG. 2C shows a plot of adjusted signal intensities vs. cycle for the A, C, T and G signals after three iterations of baseline correction for the data shown in FIG. 2A.

FIG. 3 shows a plot of mean ‘on’ and ‘off’ signal intensities per sequencing cycle for a sequencing run, wherein curves are shown for raw and corrected signal intensities.

FIG. 4 shows a plot of cumulative error versus cycle for a sequencing run, wherein curves are shown for raw and corrected signal intensities.

### DETAILED DESCRIPTION

The present disclosure provides methods for correcting imaging data, or other signal collections, acquired from nucleic acid arrays or other multiplexed analytical devices. In particular embodiments, images are obtained from an array of nucleic acids during a sequencing procedure. Signal intensities can be extracted from the images and corrected using methods set forth herein, thereby improving the quality of base calls and reduce the percent error of base calls. Accordingly, particular embodiments of the methods set forth herein can be used to analyze signals acquired from a nucleic acid sequencing system in order to improve the performance of the nucleic acid sequencing system.

Taking Sequencing By Binding™ (SBB™) technology as an example, an array of primed, genomic DNA fragments can be treated with polymerase and different nucleotide types under conditions where ternary complexes can form between a primed DNA, polymerase and next correct nucleotide. Ternary complexes can be uniquely labeled with respect to the type of nucleotide that is present in the complex. As such, images of the array acquired for each SBB™ cycle will distinguish the next correct nucleotide for each genomic DNA fragment in the array. The next correct nucleotide can be identified as the nucleotide type having the signal that has the highest intensity for that particular genomic DNA fragment for that particular cycle. The highest intensity signal type can be identified as the ‘on’ signal for the correct nucleotide type and the remaining signal types can be identified as the ‘off’ signals, where the assumption is that the ‘on’ signal intensity is larger than the ‘off’ signal intensities. The ‘off’ signal can be detected due to any number of phenomena that cause noise, drift or interference in the sequencing platform. Notably, if there is a trend where the ‘off’ signal intensities increase by different amounts for each nucleotide over multiple of cycles of the SBB™ procedure, then an incorrect base call may be made due to a nucleotide having an ‘off’ baseline intensity that is higher than a nucleotide that is ‘on’ but has a lower baseline.

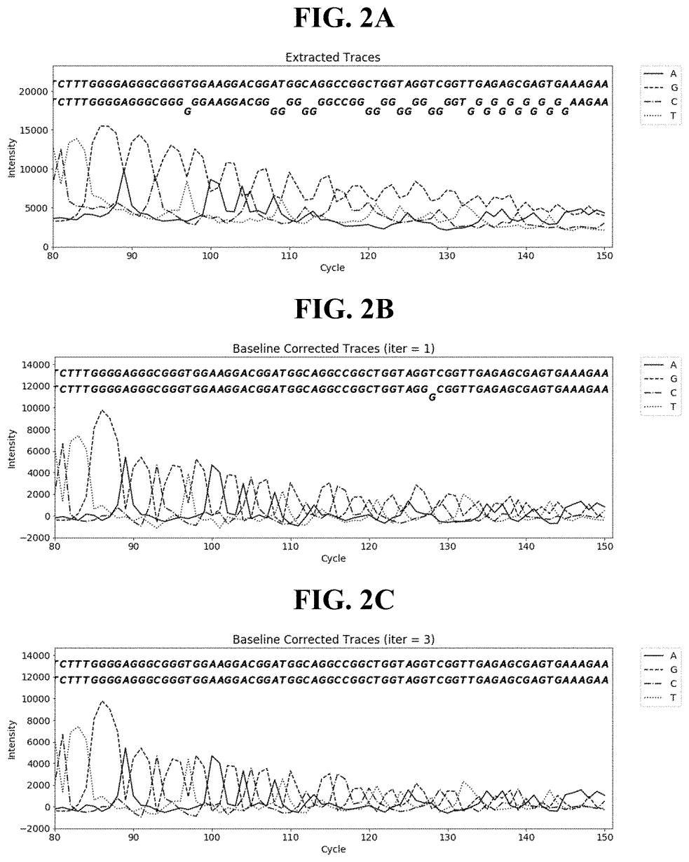

As an illustrative example, FIG. 2A shows four signal traces, each for a respective nucleotide type, all extracted from a single nucleic acid feature. The G nucleotide trace has a baseline drift that results in several miscalls wherein G is called instead of the correct nucleotide. The miscalls are identified by comparing the reference sequence (upper line in the figure) to the called sequence (lower line in the figure), the miscalls being emphasized by a subscript offset. The methods of the present disclosure are useful for correcting the baselines of the individual signal traces (i.e. correction is carried out on a feature-by-feature and nucleotide-by-nucleotide basis). As demonstrated by the results of FIG. 2B and 2C, miscalls were removed and sequencing accuracy substantially improved via iterative baseline adjustment using the methods of the present disclosure.

Other sequencing technologies also acquire image data from arrays where different nucleotide types that are present at different array features are distinguished by unique signals. Realistically, any given feature observed at any given cycle will produce an ‘on’ signal for the correct nucleotide type and ‘off’ signals that are correlated with other nucleotide types. The methods set forth herein can be used to correct image data to better distinguish ‘on’ signals from ‘off’ signals, thereby improving base calling in any of a variety of nucleic acid sequencing techniques.

The methods set forth herein are unique in providing correction for each feature in an array (or other multiplex format), each nucleotide type, and each sequencing cycle, effectively correcting every signal value being processed. In particular embodiments, baseline correction is achieved by adjusting the data with no need to make any assumption of a model or functional form for the correction. Therefore, the present methods can correct for a wide variety of sources of aberrant ‘off’ signal baseline values including, but not limited to, those situations where the root cause of noise and interference is not known.

Terms used herein will be understood to take on their ordinary meaning in the relevant art unless specified otherwise. Several terms used herein and their meanings are set forth below.

As used herein, the term “array” refers to a population of molecules that are attached to one or more solid-phase substrates such that the molecules at one feature can be distinguished from molecules at other features. An array can include different molecules that are each located at different addressable features on a solid-phase substrate. Alternatively, an array can include separate solid-phase substrates each functioning as a feature that bears a different molecule, wherein the different molecules can be identified according to the locations of the solid-phase substrates on a surface to which the solid-phase substrates are attached, or according to the locations of the solid-phase substrates in a liquid such as a fluid stream. The molecules of the array can be, for example, nucleotides, nucleic acid primers, nucleic acid templates or nucleic acid enzymes such as polymerases, ligases, exonucleases or combinations thereof.

As used herein, the term “blocking moiety,” when used in reference to a nucleotide, means a part of the nucleotide that inhibits or prevents the 3′ oxygen of the nucleotide from forming a covalent linkage to a next correct nucleotide during a nucleic acid polymerization reaction. The blocking moiety of a “reversible terminator” nucleotide can be removed from the nucleotide analog, or otherwise modified, to allow the 3′-oxygen of the nucleotide to covalently link to a next correct nucleotide. This process is referred to as “deblocking” the nucleotide analog. Such a blocking moiety is referred to herein as a “reversible terminator moiety.” Exemplary reversible terminator moieties are set forth in U.S. Pat. Nos. 7,427,673; 7,414,116; 7,057,026; 7,544,794 or 8,034,923; or PCT publications WO 91/06678 or WO 07/123744, each of which is incorporated herein by reference. A nucleotide that has a blocking moiety or reversible terminator moiety can be at the 3′ end of a nucleic acid, such as a primer, or can be a monomer that is not covalently attached to a nucleic acid.

As used herein, the term “call,” when used in reference to a nucleotide or base, refers to a determination of the type of nucleotide or base that is present at a particular position in a nucleic acid sequence. A call can be associated with a measure of error or confidence. A call of ‘N,’ ‘null,’ ‘unknown’ or the like can be used for a particular position in a sequence when an error is apparent or when confidence is below a given threshold. A call can designate a discrete type of base or nucleotide (e.g. A, C, G, T or U, using the IUPAC single letter code) or a call can designate degeneracy. Continuing with IUPAC symbols, a single position can be called as R (i.e. A or G), M (i.e. A or C), W (i.e. A or T), S (i.e. C or G), Y (i.e. C or T), K (i.e. G or T), B (i.e. C or G or T), D (i.e. A or G or T), H (i.e. A or C or T), or V (i.e. A or C or G). A call need not be final, for example, being a candidate call based on incomplete or developing information. In some cases, a call can be deemed as valid or invalid based on comparison of empirical data to a reference. For example, when signal data is encoded, a call that is consistent with a predetermined codeword for a particular base type can be identified as a valid call, whereas a call that is not consistent with codewords for any base type can be identified as an invalid call.

The term “comprising” is intended herein to be open-ended, including not only the recited elements, but further encompassing any additional elements.

The terms “cycle” or “round,” when used in reference to a sequencing procedure, refer to the portion of a sequencing run that is repeated to indicate the presence of a nucleotide. Typically, a cycle or round includes several steps such as steps for delivery of reagents, washing away unreacted reagents and detection of signals indicative of changes occurring in response to added reagents. Two cycles need not result from separate reagent deliveries. Rather, a first cycle can be completed by the same reagent mixture that completes a second cycle, for example, in a ‘single pot’ sequencing reaction.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “exogenous,” when used in reference to a moiety of a molecule, means a chemical moiety that is not present in a natural analog of the molecule. For example, an exogenous label of a nucleotide is a label that is not present on a naturally occurring nucleotide. Similarly, an exogenous label that is present on a polymerase is not found on the polymerase in its native milieu.

As used herein, the term “extension,” when used in reference to a nucleic acid, means a process of adding at least one nucleotide to the 3′ end of the nucleic acid. The term “polymerase extension,” when used in reference to a nucleic acid, refers to a polymerase catalyzed process of adding at least one nucleotide to the 3′ end of the nucleic acid. A nucleotide or oligonucleotide that is added to a nucleic acid by extension is said to be incorporated into the nucleic acid. Accordingly, the term “incorporating” can be used to refer to the process of joining a nucleotide or oligonucleotide to the 3′ end of a nucleic acid by formation of a phosphodiester bond.

As used herein, the term “extendable,” when used in reference to a nucleotide, means that the nucleotide has an oxygen or hydroxyl moiety at the 3′ position, and is capable of forming a covalent linkage to a next correct nucleotide if and when incorporated into a nucleic acid. An extendable nucleotide can be at the 3′ position of a primer or it can be a monomeric nucleotide. A nucleotide that is extendable will lack blocking moieties such as reversible terminator moieties.

As used herein, the term “extended primer hybrid” refers to a primer-template nucleic acid hybrid following incorporation of at least one nucleotide to the primer. The incorporation event can be, for example, polymerase catalyzed addition of one or more nucleotides to the 3′ end of the primer.

As used herein, the term “feature,” when used in reference to an array, means a location in an array where a particular molecule is present. A feature can contain only a single molecule or it can contain a population of several molecules of the same species (i.e. an ensemble of the molecules). Alternatively, a feature can include a population of molecules that are different species (e.g. a population of ternary complexes having different template sequences). Features of an array are typically discrete. The discrete features can be contiguous or they can have spaces between each other. An array useful herein can have, for example, features that are separated by less than 100 microns, 50 microns, 10 microns, 5 microns, 1 micron, or 0.5 micron. Alternatively or additionally, an array can have features that are separated by greater than 0.5 micron, 1 micron, 5 microns, 10 microns, 50 microns or 100 microns. The features can each have an area of less than 1 square millimeter, 500 square microns, 100 square microns, 25 square microns, 1 square micron or less.

As used herein, the term “label” refers to a molecule or moiety thereof that provides a detectable characteristic. The detectable characteristic can be, for example, an optical signal such as absorbance of radiation, fluorescence emission, luminescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like.

As used herein, the term “next correct nucleotide” refers to the nucleotide type that will bind and/or incorporate at the 3′ end of a primer to complement a base in a template strand to which the primer is hybridized. The base in the template strand is referred to as the “next base” and is immediately 5′ of the base in the template that is hybridized to the 3′ end of the primer. The next correct nucleotide can be referred to as the “cognate” of the next base and vice versa. Cognate nucleotides that interact with each other in a ternary complex or in a double stranded nucleic acid are said to “pair” with each other. A nucleotide having a base that is not complementary to the next template base is referred to as an “incorrect”, “mismatch” or “non-cognate” nucleotide.

As used herein, the term “nucleic acid sequencing procedure” refers to a process that produces a series of signals that is indicative of the sequence of nucleotides in the nucleic acid. The process can consist of repeated cycles of reagent delivery and/or detection. In some embodiments, detection is continuous. In some embodiments, multiple reaction cycles result from a single reagent delivery. Generally, signals are correlated with a particular type of nucleic acid base such that a series of signals obtained from a sequencing procedure identify the sequence of bases in the nucleic acid.

As used herein, the term “nucleotide” can be used to refer to a native nucleotide or analog thereof. Examples include, but are not limited to, nucleotide triphosphates (NTPs) such as ribonucleotide triphosphates (rNTPs), deoxyribonucleotide triphosphates (dNTPs), or non-natural analogs thereof such as dideoxyribonucleotide triphosphates (ddNTPs) or reversibly terminated nucleotide triphosphates (rtNTPs).

As used herein, the term “polymerase” can be used to refer to a nucleic acid synthesizing enzyme, including but not limited to, DNA polymerase, RNA polymerase, reverse transcriptase, primase and transferase. Typically, the polymerase has one or more active sites at which nucleotide binding and/or catalysis of nucleotide polymerization may occur. The polymerase may catalyze the polymerization of nucleotides to the 3′ end of the first strand of the double stranded nucleic acid molecule. For example, a polymerase catalyzes the addition of a next correct nucleotide to the 3′ oxygen group of the first strand of the double stranded nucleic acid molecule via a phosphodiester bond, thereby covalently incorporating the nucleotide to the first strand of the double stranded nucleic acid molecule. Optionally, a polymerase need not be capable of nucleotide incorporation under one or more conditions used in a method set forth herein. For example, a mutant polymerase may be capable of forming a ternary complex but incapable of catalyzing nucleotide incorporation.

As used herein, the term “primer-template nucleic acid hybrid” or “primer-template hybrid” refers to a nucleic acid hybrid having a double stranded region such that one of the strands has a 3′-end that can be extended by a polymerase. The two strands can be parts of a contiguous nucleic acid molecule (e.g. a hairpin structure) or the two strands can be separable molecules that are not covalently attached to each other.

As used herein, the term “primer” refers to a nucleic acid having a sequence that binds to a nucleic acid at or near a template sequence. Generally, the primer binds in a configuration that allows replication of the template, for example, via polymerase extension of the primer. The primer can be a first portion of a nucleic acid molecule that binds to a second portion of the nucleic acid molecule, the first portion being a primer sequence and the second portion being a primer binding sequence (e.g. a hairpin primer). Alternatively, the primer can be a first nucleic acid molecule that binds to a second nucleic acid molecule having the template sequence. A primer can consist of DNA, RNA or analogs thereof.

As used herein, the term “signal” refers to energy or coded information that can be selectively observed over other energy or information such as background energy or information. A signal can have a desired or predefined characteristic. For example, an optical signal can be characterized or observed by one or more of intensity, wavelength (e.g. color), energy, frequency, power, lifetime, luminance or the like. Other signals can be quantified according to characteristics such as voltage, current, electric field strength, magnetic field strength, frequency, power, temperature, etc. An optical signal can be detected at a particular intensity, wavelength, or color; an electrical signal can be detected at a particular frequency, power or field strength; or other signals can be detected based on characteristics known in the art pertaining to spectroscopy and analytical detection. Absence of signal is understood to be a signal level of zero or a signal level that is not meaningfully distinguished from noise.

As used herein, the term “signal trace” can refer to a structure or representation of nucleic acid sequencing data that correlates each sequencing cycle with one or more signal characteristics acquired for the cycle. For example, a signal trace can correlate signal characteristics with sequencing cycles for a particular feature in an array of nucleic acids that is subjected to the sequencing cycles. Optionally, the signal trace can correlate signals for one type of nucleotide with each cycle. In some configurations a signal trace can be represented as a plot of signal characteristics vs. cycle. Other representations can be used including, for example, a table, list or other computer readable data structure.

As used herein, the term “ternary complex” refers to an intermolecular association between a polymerase, a double stranded nucleic acid and a nucleotide. Typically, the polymerase facilitates interaction between a next correct nucleotide and a template strand of the primed nucleic acid. A next correct nucleotide can interact with the template strand via Watson-Crick hydrogen bonding. The term “stabilized ternary complex” means a ternary complex having promoted or prolonged existence or a ternary complex for which disruption has been inhibited. Generally, stabilization of the ternary complex prevents covalent incorporation of the nucleotide component of the ternary complex into the primed nucleic acid component of the ternary complex.

As used herein, the term “type” or “species” is used to identify molecules that share the same chemical structure. For example, a mixture of nucleotides can include several dCTP molecules. The dCTP molecules will be understood to be the same type (or species) as each other, but a different type (or species) compared to dATP, dGTP, dTTP etc. Similarly, individual DNA molecules that have the same sequence of nucleotides are the same type (or species), whereas DNA molecules with different sequences are different types (or species). The term “type” or “species” can also identify moieties that share the same chemical structure. For example, the cytosine bases in a template nucleic acid will be understood to be the same type (or species) of base as each other independent of their position in the template sequence.

The embodiments set forth below and recited in the claims can be understood in view of the above definitions.

The present disclosure provides a method of determining nucleic acid sequences. The method can include steps of (a) obtaining signal data from a nucleic acid sequencing procedure carried out on an array of nucleic acid features; (b) extracting signals from each nucleic acid feature to produce multiple extracted signal traces, wherein each extracted signal trace correlates signal characteristics with sequencing cycle for a particular nucleotide type at a particular nucleic acid feature; (c) comparing the extracted signal traces for different nucleotide types at each of the features, thereby distinguishing an extracted signal having a characteristic of a candidate base call from extracted background signals for each cycle at each feature; (d) applying a baseline adjustment to each extracted signal trace based on the extracted background signals, thereby obtaining a adjusted signal trace for each nucleotide at each feature; and (e) comparing the adjusted signal traces for different nucleotide types at each of the features, thereby distinguishing adjusted signals having characteristics of a base call from adjusted background signals for each cycle at each feature, whereby nucleic acid sequences are determined from the sequence of the base calls at each of the features. The method can obtain the signal data from a nucleic acid sequencing system and can be used to improve the signal to noise, base call accuracy and/or read length of the sequencing system.

In particular embodiments, primary signals are distinguished from background signals for each feature of an array and for each cycle of a sequencing protocol carried out on the array. A primary signal is distinguished from background signals based on a characteristic that is indicative of the type of nucleotide that is present at a particular position of a target nucleic acid that is being sequenced. For example, when different nucleotide types are correlated with different luminescence colors (i.e. emission wavelengths), the color having the highest intensity of emission at a particular feature for a particular cycle can be identified as the primary signal. Signals for all other nucleotide types are identified as background signals for that particular feature at that particular cycle.

Signal intensity distinction is useful for example when evaluating SBB™ or SBS sequencing protocols. Other signal characteristics can be used to distinguish a primary signal from background signals in accordance with the detection modalities used for particular sequencing protocols. For example, a primary signal can be the signal type having the longest duration (e.g. in the case of sequencing protocols that detect residence time for nucleotide, polymerase or other sequencing reagents at an array feature), the largest magnitude of a shift in wavelength (e.g. in the case of sequencing protocols that detect chromatic shifts, Forster resonance energy transfer etc.), the lowest intensity signal (e.g. when detection is based on quenching a label), or the shortest duration (e.g. when detection is based on displacement of a label). The primary signal may be referred to as the ‘on’ signal and background signals may be referred to as ‘off’ signals.

Typically, a sequencing procedure will be capable of distinguishing four nucleotide types by detecting four different signal types. Accordingly, an individual feature in an array can produce a primary signal that is indicative of one type of nucleotide and that is distinguished from three background signals that are correlated with three other types of nucleotides. Often, the primary signal and one or more of the background signals are observed at the feature. It will be understood that, depending upon the sequencing protocol used, fewer than 4 signal types can be used to distinguish nucleotide types. For example, detectable signals may be produced by at most 3, 2 or 1 nucleotide types. Such methods are said to utilize a ‘dark’ base, wherein the presence of at least one base type is imputed from absence of a signal. Alternatively or additionally, more than 4 signal types can be used to distinguish nucleotide types. For example, an individual nucleotide type can be encoded by at least 2, 3 or 4 different signal types as set forth in U.S. patent application Ser. No. 15/922,787, now granted as U.S. Pat. No. 10,161,003, each of which is incorporated herein by reference. Exemplary protocols that use varying numbers of signal types to distinguish different nucleotide types are set forth in U.S. patent application Ser. No. 15/712,632, now granted as U.S. Pat. No. 9,951,385; U.S. Pat. No. 9,523,125; or U.S. Pat. No. 9,453,258, each of which is incorporated herein by reference.

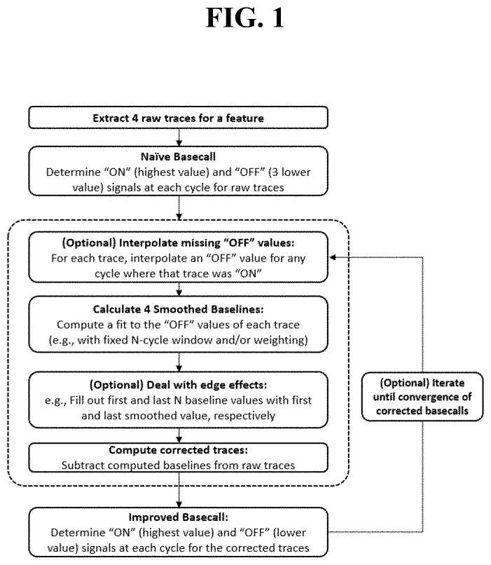

An exemplary embodiment for processing signals in order to make base calls is diagrammed in FIG. 1. For clarity of description, the process is described for a single feature on an array. However, the process is generally applicable to a plurality of features. Each feature in an array can be individually processed (in parallel or sequentially) as exemplified for one feature. In the first step, images are obtained from an array. In this example, four different nucleotides are distinguished (e.g. due to unique labeling or unique timing of delivery and detection during the sequencing cycle), and each nucleotide type is detected in one of four raw images. The signals for each feature of the array and for each nucleotide type can be represented as a trace that correlates signal intensity with cycle number.

In the second step, a naive base call is made for each cycle based on relative signal intensities whereby the signal trace with the highest raw intensity at a particular cycle is identified as the trace for the candidate ‘on’ nucleotide type for that feature at that cycle. The other nucleotide types are candidate ‘off’ nucleotides for that feature at that cycle.

Continuing with the embodiment of FIG. 1, the baseline is corrected using the four sub-steps shown in the dashed-line box. The first sub-step involves interpolating missing ‘off’ signal intensities for the raw signal traces. For example, the first sub-step can be carried out by applying a linear interpolation function to the raw signal traces, thereby producing linearly interpolated signal traces for the feature and for the nucleotide type. The second sub-step is to apply a smoothing function to the raw (optionally interpolated) signal traces. The smoothing function can use a fixed window size and/or fixed weighting for ‘off’ signal intensities. The ‘on’ signals are omitted from the smoothing function. The third sub-step is to optionally fix edge effects, for example, by filling in beginning and end of cycles in the window with a first and last smoothed value, respectively. The fourth step is to compute corrected signal traces by subtracting the computed baseline from the raw signal traces. The computed ‘off’ baseline is subtracted from all signal intensities (both ‘on’ and ‘off’ signals) in the raw traces.

In the next step of FIG. 1, improved base calls are made based on relative signal intensities whereby the corrected signal trace with the highest raw intensity at a particular cycle for a particular feature is identified as the trace for the candidate ‘on’ nucleotide type for that cycle and that feature and all other nucleotide types are candidate ‘off’ nucleotides for that cycle and that feature. Optionally, an iteration is carried out whereby the corrected signal trace is subjected to the four sub-steps for computing a corrected baseline. Iteration can be carried out until convergence is observed. As a result, a final base call is made for each cycle at each cluster based on the largest difference between the ‘on’ signal and the ‘off’ signals for that cycle in the baseline corrected traces for that cluster.

Any of a variety of algorithms can be used to adjust signal traces. Generally, signals are sorted such that ‘off’ signals are used as a basis for the adjustment. For example, signal intensity values that are identified as ‘off’ signals (e.g. background signals) are used as a basis for smoothing or adjusting signal traces. The signals that are identified as ‘on’ signals (e.g. candidate base calls) can be omitted from calculations that are used to adjust or smooth a signal trace.

Smoothing is a low-pass filter that can be used for removing high-frequency noise from signal traces. Smoothing can be based on an assumption that signals which are near to each other in a signal trace can be averaged together to reduce noise without significant loss of the signal of interest. In some embodiments, boxcar averaging can be used to enhance signal-to-noise of a signal trace by replacing a window of consecutive data points with its average. A modified smoothing approach can use weighted points in the window of consecutive data points. Weighting can be symmetric or asymmetric as desired to suit a particular signal type or sequencing condition.

A further exemplary smoothing algorithm is the Savitzky-Golay algorithm (Savitzky and Golay Anal. Chem., 36, pp 1627-1639 (1964), which is incorporated herein by reference). The algorithm can be used to fit individual polynomials to windows around each signal in a signal trace. These polynomials are then used to smooth the data. The algorithm returns results based on selection of both the size of the window (filter width) and the order of the polynomial. The larger the window and the lower the polynomial order, the more smoothing that occurs. Typically, the window will be selected to be on the order of, or smaller than, the nominal width of non-noise features.

Derivatives are useful for removing unimportant baseline signal from signal traces by taking the derivative of the measured signal characteristics (e.g. signal intensity) with respect to cycle number. Derivatives are a form of high-pass filter and frequency-dependent scaling and can be used when lower-frequency (i.e., smooth and broad) features in the trace, such as baselines, are interferences, and when higher-frequency (i.e., sharp and narrow) features in the trace contain signals of interest. A relatively simple form of derivative is a point-difference first derivative, in which each signal in a signal trace is subtracted from its immediate neighboring signal. This subtraction removes the signal which is the same between the two variables and leaves only the part of the signal which is different. When performed on an entire signal trace, a first derivative can effectively remove any offset from baseline and de-emphasize lower-frequency signals. A second derivative can be calculated by repeating the process, which will further accentuate higher-frequency features in the signal trace.

Another useful derivative subtracts a signal obtained for one nucleotide type from signal(s) obtained for at least one other nucleotide type at a particular nucleic acid feature during a particular cycle. This type of derivative can be useful, for example, when nucleotides are present in various combinations during a sequencing cycle. Nucleotide combinations can result from simultaneous delivery of the combined nucleotides. Alternatively, a nucleotide combination can result from sequential addition of nucleotides such that a first nucleotide type is not removed until after one or more other nucleotide(s) have been delivered and the resulting combination detected.

Because derivatives de-emphasize lower frequencies and emphasize higher frequencies, they tend to accentuate noise (high frequency signal). For this reason, the Savitzky-Golay algorithm can be used to simultaneously smooth the data as it takes the derivative, thereby improving base calls made from the derivatized data. As with smoothing, the Savitzky-Golay derivatization algorithm returns results based on the size of the window (filter width), the order of the polynomial, and the order of the derivative. The larger the window and the lower the polynomial order, the more smoothing that occurs. Typically, the window will be on the order of, or smaller than, the nominal width of non-noise features in a signal trace which should not be smoothed.

A detrend algorithm can be particularly useful for signal traces having a constant, linear, or curved offset. Detrend can be used to fit a polynomial of a given order to the entire signal trace and simply subtracts this polynomial. This algorithm fits the polynomial to all points in a signal trace, baseline and signal of interest. As such, this method is particularly useful when the largest source of signal in each signal trace is background interference.

A Specified Points Baseline algorithm can be used to fit a polynomial of a specific order to points in a signal trace which are known to be baseline (‘off’ signal) points. This method can be useful when the signal in some signal traces is due only to background. These variables serve as good references for how much background should be removed from nearby variables.

Another algorithm that can be used to automatically remove baseline offsets from data uses the Weighted Least Squares (WLS) method. This method is useful when the signal for some cycles in a trace are due only to ‘off’ signals. These variables serve as good references for how much background should be removed from nearby variables. The WLS algorithm can use an automatic approach to determine which points in a signal trace are most likely due to ‘off’ signals alone. This can be achieved by iteratively fitting a baseline to each signal trace and determining which variables are clearly above the baseline (i.e., ‘on’ signal) and which are below the baseline. The points below the baseline are assumed to be more significant in fitting the baseline to the signal trace. This method is also called asymmetric weighted least squares. The net effect is an automatic removal of background while avoiding the creation of highly negative peaks. Typically, the baseline is approximated by some low-order polynomial, but one or more specific baseline references can be supplied. When specific references are provided as the basis, the background will be removed by subtracting some amount of each of these references to obtain a low background result without negative peaks.

It will be understood that signal traces can also be normalized on a feature-by-feature and nucleotide type-by-nucleotide type basis. The algorithm can function similarly to the baseline adjustment algorithms exemplified above, except that (1) the signals are sorted to identify the ‘on’ signals that will be used for normalization and to omit the ‘off’ signals from the normalization; and (2) the raw signal traces are divided by the ‘on’ signals that have been sorted out. Normalization can be performed in addition to background adjustment or as an alternative to background adjustment.

A baseline adjustment algorithm, or other algorithm set forth herein, can be performed following completion of a sequencing run. As such, a signal trace that is used in a method or apparatus set forth herein can include signals from all cycles that are to be evaluated. Alternatively, the signals can be processed in real time or near real time as the chemical steps of sequencing are being carried out. For example, a baseline adjustment algorithm that uses a particular window size or group of signal characteristics can be initiated once sufficient cycles have been performed. More specifically, a smoothing function that utilizes a window size of 9 cycles can be initiated once the 9^{th} cycle is complete. Smoothing can continue using a sliding window whereby the signal data from the first cycle is removed from buffer storage that is used for the smoothing calculation (e.g. the data can be deleted, processed or stored in a separate memory location) and signal data from a 10^{th} cycle is added to the buffer storage for use in the calculation.

Particularly useful sequencing reactions that can be used in a method set forth herein are Sequencing By Binding™ (SBB™) reactions including, for example, those described in commonly owned US Pat. App. Pub. No. 2017/0022553 A1; or U.S. patent application Ser. No. 15/712,632, granted as U.S. Pat. No. 9,951,385; US Pat. App. Pub. No. 2018/0044727, which claims priority to U.S. Pat. App. Ser. No. 62/447,319; US Pat. App. Pub. No. 2018/0187245, which claims priority to U.S. Pat. App. Ser. No. 62/440,624; or US Pat. App. Pub. No. 2018/0208983, which claims priority to U.S. Pat. App. Ser. No. 62/450,397, each of which is incorporated herein by reference. Generally, methods for determining the sequence of a template nucleic acid molecule can be based on formation of a ternary complex (between polymerase, primed nucleic acid and cognate nucleotide) under specified conditions. The method can include an examination phase followed by a nucleotide incorporation phase.

The examination phase can be carried out in a flow cell (or other vessel), the flow cell containing at least one template nucleic acid molecule primed with a primer by delivering, to the flow cell, reagents to form a first reaction mixture. The reaction mixture can include the primed template nucleic acid, a polymerase and at least one nucleotide type. Interaction of polymerase and a nucleotide with the primed template nucleic acid molecule(s) can be observed under conditions where the nucleotide is not covalently added to the primer(s); and the next base in each template nucleic acid can be identified using the observed interaction of the polymerase and nucleotide with the primed template nucleic acid molecule(s). The interaction between the primed template, polymerase and nucleotide can be detected in a variety of schemes. For example, the nucleotides can contain a detectable label. Each nucleotide can have a distinguishable label with respect to other nucleotides. Alternatively, some or all of the different nucleotide types can have the same label and the nucleotide types can be distinguished based on separate deliveries of different nucleotide types to the flow cell. In some embodiments, the polymerase can be labeled. Polymerases that are associated with different nucleotide types can have unique labels that distinguish the type of nucleotide to which they are associated. Alternatively, polymerases can have similar labels and the different nucleotide types can be distinguished based on separate deliveries of different nucleotide types to the flow cell. Signals can be obtained using methods appropriate for the labels used. The signals can be processed using methods set forth herein to correct signal traces, or to adjust for noise or interference.

During the examination phase, discrimination between correct and incorrect nucleotides can be facilitated by ternary complex stabilization. A variety of conditions and reagents can be useful. For example, the primer can contain a reversible blocking moiety that prevents covalent attachment of nucleotide; and/or cofactors that are required for extension, such as divalent metal ions, can be absent; and/or inhibitory divalent cations that inhibit polymerase-based primer extension can be present; and/or the polymerase that is present in the examination phase can have a chemical modification and/or mutation that inhibits primer extension; and/or the nucleotides can have chemical modifications that inhibit incorporation, such as 5′ modifications that remove or alter the native triphosphate moiety.

The extension phase can be carried out after examination by creating conditions in the flow cell (or other reaction vessel) where a nucleotide can be added to the primer on each template nucleic acid molecule. In some embodiments, this involves removal of reagents used in the examination phase and replacing them with reagents that facilitate extension. For example, examination reagents can be replaced with a polymerase and nucleotide(s) that are capable of extension. Alternatively, one or more reagents can be added to the examination phase reaction to create extension conditions. For example, catalytic divalent cations can be added to an examination mixture that was deficient in the cations, and/or polymerase inhibitors can be removed or disabled, and/or extension competent nucleotides can be added, and/or a deblocking reagent can be added to render primer(s) extension competent, and/or extension competent polymerase can be added. The extension step can be carried out with nucleotides that are unlabeled. The nucleotides, whether labeled or not, can include a reversible terminator moiety.

Accordingly, a Sequencing by Binding method can include steps of (a) obtaining signal data from a nucleic acid sequencing procedure carried out on an array of nucleic acid features, wherein the sequencing procedure includes steps of: (i) contacting the array with reagents for forming ternary complexes, wherein the reagents include a polymerase and nucleotide cognates for at least three different base types suspected of being present in the nucleic acids; (ii) acquiring signals from the features while precluding polymerase catalyzed extension of the nucleic acids at the features; and (iii) after step (ii), extending the nucleic acids at the features, wherein different nucleotide types produce different signals, and wherein each feature produces a primary signal indicative of one type of nucleotide and secondary signals indicative of other types of nucleotides; (b) extracting signals from each nucleic acid feature to produce multiple extracted signal traces, wherein each extracted signal trace correlates signal characteristics with sequencing cycle for a particular nucleotide type at a particular nucleic acid feature; (c) comparing the extracted signal traces for different nucleotide types at each of the features, thereby distinguishing an extracted signal having a characteristic of a candidate base call from extracted background signals for each cycle at each feature; (d) applying a baseline adjustment to each extracted signal trace based on the extracted background signals, thereby obtaining a adjusted signal traces for each nucleotide at each feature; and (e) comparing the adjusted signal traces for different nucleotide types at each of the features, thereby distinguishing adjusted signals having characteristics of a base call from adjusted background signals for each cycle at each feature, whereby nucleic acid sequences are determined from the sequence of the base calls at each of the features.

Generally for SBB™ embodiments, the primary (or ‘on’) signal is produced by ternary complex comprising the next correct nucleotide. The background (or ‘off’) signals are typically produced by non-specific interactions of labeled reagents with the features. Other mechanisms such as phasing, detection channel crosstalk and the like may also contribute to the presence of ‘off’ signals. An advantage of the baseline correction methods is that the mechanism need not be known in order to achieve the correction.

Sequencing-by-synthesis (SBS) techniques can be used. SBS generally involves the enzymatic extension of a nascent primer through the iterative addition of nucleotides against a template strand to which the primer is hybridized. Briefly, SBS can be initiated by contacting target nucleic acids, attached to sites (e.g. arrayed features) in a vessel, with one or more labeled nucleotides, DNA polymerase, etc. Those features where a primer is extended using the target nucleic acid as template will incorporate a labeled nucleotide that can be detected. Detection can include scanning using an apparatus or method set forth herein. Optionally, the labeled nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove or modify the moiety. Thus, for embodiments that use reversible termination, a deblocking reagent can be delivered to the vessel (before or after detection occurs). Washes can be carried out between the various delivery steps. The cycle can be performed n times to extend the primer by n nucleotides, thereby detecting a sequence of length n. Exemplary SBS procedures, reagents and detection components that can be readily adapted for use in the methods of the present disclosure are described, for example, in Bentley et al., Nature 456:53-59 (2008), WO 04/018497; WO 91/06678; WO 07/123744; U.S. Pat. Nos. 7,057,026; 7,329,492; 7,211,414; 7,315,019 or 7,405,281, and US Pat. App. Pub. No. 2008/0108082 A1, each of which is incorporated herein by reference. Also useful are SBS methods that are commercially available from Illumina, Inc. (San Diego, Calif.).

Signals obtained from an SBS method can be corrected using methods set forth herein. For example, signals that are obtained from an array can be classified such that the highest intensity signal is generally identified as the correct nucleotide (or ‘on’ signal) and other signals are identified as incorrect nucleotides (or ‘off’ signals). The ‘off’ signals can be used in methods set forth herein to correct signal traces that have been extracted for individual nucleotide types at individual clusters (or at other array features used in the sequencing protocol). As such, the methods can correct for stochastic errors, phasing errors or other errors.

Accordingly, a Sequencing by Synthesis method can include steps of (a) obtaining signal data from a nucleic acid sequencing procedure carried out on an array of nucleic acid features, wherein the sequencing procedure includes steps of: (i) contacting the array with reagents for adding a labeled nucleotide to the 3′ end of a nucleic acid at each of the features; and (ii) acquiring signals from the labeled nucleotides added at the features, wherein different nucleotide types produce different signals, and wherein each feature produces a primary signal indicative of one type of nucleotide and secondary signals indicative of other types of nucleotides; (b) extracting signals from each nucleic acid feature to produce multiple extracted signal traces, wherein each extracted signal trace correlates signal characteristics with sequencing cycle for a particular nucleotide type at a particular nucleic acid feature; (c) comparing the extracted signal traces for different nucleotide types at each of the features, thereby distinguishing an extracted signal having a characteristic of a candidate base call from extracted background signals for each cycle at each feature; (d) applying a baseline adjustment to each extracted signal trace based on the extracted background signals, thereby obtaining a adjusted signal trace for each nucleotide at each feature; and (e) comparing the adjusted signal traces for different nucleotide types at each of the features, thereby distinguishing adjusted signals having characteristics of a base call from adjusted background signals for each cycle at each feature, whereby nucleic acid sequences are determined from the sequence of the base calls at each of the features.

Some SBS embodiments include detection of a proton released upon incorporation of a nucleotide into an extension product. For example, sequencing based on detection of released protons can use reagents and an electrical detector that are commercially available from ThermoFisher (Waltham, Mass.) or described in US Pat. App. Pub. Nos. 2009/0026082 A1; 2009/0127589 A1; 2010/0137143 A1; or 2010/0282617 A1, each of which is incorporated herein by reference. In such embodiments, protons released from the correct nucleotide will generally produce the highest signal intensity and can be identified as ‘on’ signals, whereas other signals can be identified as ‘off’ signals. The ‘off’ signals can be used in methods set forth herein to correct signal traces that have been extracted for individual nucleotide types at individual clusters (or at other array features used in the sequencing protocol).

Other sequencing procedures can be used, such as pyrosequencing. Pyrosequencing detects the release of inorganic pyrophosphate (PPi) as nucleotides are incorporated into a nascent primer hybridized to a template nucleic acid strand (Ronaghi, et al., Analytical Biochemistry 242 (1), 84-9 (1996); Ronaghi, Genome Res. 11 (1), 3-11 (2001); Ronaghi et al. Science 281 (5375), 363 (1998); U.S. Pat. Nos. 6,210,891; 6,258,568 and 6,274,320, each of which is incorporated herein by reference). In pyrosequencing, released PPi can be detected by being converted to adenosine triphosphate (ATP) by ATP sulfurylase, and the resulting ATP can be detected via luciferase-produced photons. Luminescent signals produced from incorporation of the correct nucleotide will generally produce the highest signal intensity and can be identified as ‘on’ signals, whereas other signals can be identified as ‘off’ signals. The ‘off’ signals can be used in methods set forth herein to correct signal traces that have been extracted for individual nucleotide types at individual clusters (or at other array features used in the sequencing protocol).

Sequencing-by-ligation reactions are also useful including, for example, those described in Shendure et al. Science 309:1728-1732 (2005); U.S. Pat. Nos. 5,599,675; or 5,750,341, each of which is incorporated herein by reference. Some embodiments can include sequencing-by-hybridization procedures as described, for example, in Bains et al., Journal of Theoretical Biology 135 (3), 303-7 (1988); Drmanac et al., Nature Biotechnology 16, 54-58 (1998); Fodor et al., Science 251 (4995), 767-773 (1995); or WO 1989/10977, each of which is incorporated herein by reference. In both sequencing-by-ligation and sequencing-by-hybridization procedures, primers that are hybridized to nucleic acid templates are subjected to repeated cycles of extension by oligonucleotide ligation. Typically, the oligonucleotides are fluorescently labeled and can be detected to determine the sequence of the template. Signals detected from oligonucleotides having the correct nucleotide will generally produce the highest signal intensity and can be identified as ‘on’ signals, whereas other signals can be identified as ‘off’ signals. The ‘off’ signals can be used in methods set forth herein to correct signal traces that have been extracted for individual nucleotide types at individual clusters (or at other array features used in the sequencing protocol).

Some embodiments can utilize methods involving real-time monitoring of DNA polymerase activity. For example, nucleotide incorporations can be detected through fluorescence resonance energy transfer (FRET) interactions between a fluorophore-bearing polymerase and gamma-phosphate-labeled nucleotides, or with zero-mode waveguides (ZMW). Techniques and reagents for sequencing via FRET and or ZMW detection that can be modified for use in an apparatus or method set forth herein are described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008); or U.S. Pat. Nos. 7,315,019; 8,252,911 or 8,530,164, the disclosures of which are incorporated herein by reference. Baselines for signals detected by an array of ZMWs can be corrected using methods set forth herein. Generally, for methods that use a ZMW to detect interactions between a gamma phosphate labeled nucleotide and polymerase, primary signals can be identified as those having longer duration and shorter duration signals can be identified as secondary signals. In the case of FRET-based sequencing methods, ‘on’ signals can be distinguished from ‘off’ signals based on the magnitude of wavelength shifts or based on intensity of a shifted signal. The ‘off’ signals can be used in methods set forth herein to correct signal traces that have been extracted for individual nucleotide types at individual clusters (or at other array features used in the sequencing protocol).

Steps for sequencing methods can be performed cyclically. For example, examination and extension steps of an SBB method can be repeated such that in each cycle a single next correct nucleotide is examined (i.e. the next correct nucleotide being a nucleotide that correctly binds to the nucleotide in a template nucleic acid that is located immediately 5′ of the base in the template that is hybridized to the 3′-end of the hybridized primer) and, subsequently, a single next correct nucleotide is added to the primer. Any number of cycles of a sequencing method set forth herein can be carried out including, for example, at least 1, 2, 5, 10, 20, 25, 30, 40, 50, 75, 100, 150 or more cycles. Alternatively or additionally, no more than 150, 100, 75, 50, 40, 30, 25, 20, 10, 5, 2 or 1 cycles are carried out. A trace that is generated from a sequencing method can include a data point for each cycle. As such, the number of points in a trace for a particular nucleic acid can be equivalent to the number of cycles used to sequence the nucleic acid. Multiple traces can be obtained from each of the nucleic acids. For example, an individual trace can be obtained for each nucleotide type that is suspected of being present in the nucleic acids. In configurations in which each of four nucleotide types are observed via a unique signal, four traces can be obtained from a single nucleic acid, each trace having a number of points that is equivalent to the number of sequencing cycles performed, and the four traces can be combined to determine the sequence for the nucleic acid.

Nucleic acid template(s), to be sequenced, can be added to a vessel using any of a variety of known methods. In some embodiments, a single nucleic acid molecule is to be sequenced. The nucleic acid molecule can be delivered to a vessel and can optionally be attached to a surface in the vessel. In some embodiments, the molecule is subjected to single molecule sequencing. Alternatively, multiple copies of the nucleic acid can be made, and the resulting ensemble can be sequenced. For example, the nucleic acid can be amplified on a surface (e.g. on the inner wall of a flow cell) using techniques set forth in further detail below. The resulting ensemble can be referred to as a ‘cluster’ on the surface.

In multiplex embodiments, a variety of different nucleic acid molecules (i.e. a population having a variety of different sequences) are sequenced. The molecules can optionally be attached to a surface in a vessel. The nucleic acids can be attached at unique features on the surface and single nucleic acid molecules that are spatially distinguishable one from the other can be sequenced in parallel. Alternatively, the nucleic acids can be amplified on the surface to produce a plurality of surface attached ensembles (or clusters). The ensembles function as arrayed features that can be spatially distinguishable and sequenced in parallel.

A method set forth herein can use any of a variety of amplification techniques. Exemplary techniques that can be used include, but are not limited to, polymerase chain reaction (PCR), rolling circle amplification (RCA), multiple displacement amplification (MDA), bridge amplification, or random prime amplification (RPA). In particular embodiments, one or more primers used for amplification can be attached to a surface in a vessel, such as a flow cell. Methods that result in one or more features on a solid support, where each feature is attached to multiple copies of a particular nucleic acid template, can be referred to as ‘clustering’ methods.

In PCR embodiments, one or both primers used for amplification can be attached to a surface. Formats that utilize two species of attached primer are often referred to as bridge amplification because double stranded amplicons form a bridge-like structure between the two attached primers that flank the template sequence that has been copied. Exemplary reagents and conditions that can be used for bridge amplification are described, for example, in U.S. Pat. Nos. 5,641,658 or 7,115,400; U.S. Patent Pub. Nos. 2002/0055100 A1, 2004/0096853 A1, 2004/0002090 A1, 2007/0128624 A1 or 2008/0009420 A1, each of which is incorporated herein by reference. PCR amplification can also be carried out with one of the amplification primers attached to the surface and the second primer in solution. An exemplary format that uses a combination of one solid phase-attached primer and a solution phase primer is known as primer walking and can be carried out as described in U.S. Pat. No. 9,476,080, which is incorporated herein by reference. Another example is emulsion PCR which can be carried out as described, for example, in Dressman et al., Proc. Natl. Acad. Sci. USA 100:8817-8822 (2003), WO 05/010145, or U.S. Patent Pub. Nos. 2005/0130173 A1 or 2005/0064460 A1, each of which is incorporated herein by reference.

RCA techniques can be used in a method set forth herein. Exemplary reagents that can be used in an RCA reaction and principles by which RCA produces amplicons are described, for example, in Lizardi et al., Nat. Genet. 19:225-232 (1998) or US Pat. App. Pub. No. 2007/0099208 A1, each of which is incorporated herein by reference. Primers used for RCA can be in solution or attached to a surface in a flow cell.

MDA techniques can also be used in a method of the present disclosure. Some reagents and useful conditions for MDA are described, for example, in Dean et al., Proc Natl. Acad. Sci. USA 99:5261-66 (2002); Lage et al., Genome Research 13:294-307 (2003); Walker et al., Molecular Methods for Virus Detection, Academic Press, Inc., 1995; Walker et al., Nucl. Acids Res. 20:1691-96 (1992); or U.S. Pat. Nos. 5,455,166; 5,130,238; or 6,214,587, each of which is incorporated herein by reference. Primers used for MDA can be in solution or attached to a surface in a vessel.

In particular embodiments, a combination of two or more of the above-exemplified amplification techniques can be used. For example, RCA and MDA can be used in a combination wherein RCA is used to generate a concatemeric amplicon in solution (e.g. using solution-phase primers). The amplicon can then be used as a template for MDA using primers that are attached to a surface in a vessel. In this example, amplicons produced after the combined RCA and MDA steps will be attached in the vessel. The amplicons will generally contain concatemeric repeats of a target nucleotide sequence.

Nucleic acid templates that are used in a method or composition herein can be DNA such as genomic DNA, synthetic DNA, amplified DNA, complementary DNA (cDNA) or the like. RNA can also be used such as mRNA, ribosomal RNA, tRNA or the like. Nucleic acid analogs can also be used as templates herein. Thus, a mixture of nucleic acids used herein can be derived from a biological source, synthetic source or amplification procedure. Primers used herein can be DNA, RNA or analogs thereof.

Exemplary organisms from which nucleic acids can be derived include, for example, a mammal such as a rodent, mouse, rat, rabbit, guinea pig, ungulate, horse, sheep, pig, goat, cow, cat, dog, primate, human or non-human primate; a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum. Nucleic acids can also be derived from a prokaryote such as a bacterium, Escherichia coli, staphylococci or Mycoplasma pneumoniae; an archae; a virus such as Hepatitis C virus or human immunodeficiency virus; or a viroid. Nucleic acids can be derived from a homogeneous culture or population of the above organisms or alternatively from a collection of several different organisms, for example, in a community or ecosystem. Nucleic acids can be isolated using methods known in the art including, for example, those described in Sambrook et al., Molecular Cloning: A Laboratory Manual, 3rd edition, Cold Spring Harbor Laboratory, New York (2001) or in Ausubel et al., Current Protocols in Molecular Biology, John Wiley and Sons, Baltimore, Md. (1998), each of which is incorporated herein by reference.

A template nucleic acid can be obtained from a preparative method such as genome isolation, genome fragmentation, gene cloning and/or amplification. The template can be obtained from an amplification technique such as polymerase chain reaction (PCR), rolling circle amplification (RCA), multiple displacement amplification (MDA) or the like. Exemplary methods for isolating, amplifying and fragmenting nucleic acids to produce templates for analysis are set forth in U.S. Pat. Nos. 6,355,431 or 9,045,796, each of which is incorporated herein by reference. Amplification can also be carried out using a method set forth in Sambrook et al., Molecular Cloning: A Laboratory Manual, 3rd edition, Cold Spring Harbor Laboratory, New York (2001) or in Ausubel et al., Current Protocols in Molecular Biology, John Wiley and Sons, Baltimore, Md. (1998), each of which is incorporated herein by reference.

A method of the present disclosure can be carried out for an array of features, for example, wherein each feature includes a nucleic acid. Arrays provide the advantage of facilitating multiplex detection. For example, different analytes (e.g. cells, nucleic acids, proteins, candidate small molecule therapeutics etc.) can be attached to an array via linkage of each different analyte to a particular feature of the array. Exemplary array substrates that can be useful include, without limitation, a BeadChip™ Array available from Illumina, Inc. (San Diego, Calif.) or arrays such as those described in U.S. Pat. Nos. 6,266,459; 6,355,431; 6,770,441; 6,859,570; or 7,622,294; or PCT Publication No. WO 00/63437, each of which is incorporated herein by reference. Further examples of commercially available array substrates that can be used include, for example, an Affymetrix GeneChip™ array. A spotted array substrate can also be used according to some embodiments. An exemplary spotted array is a CodeLink™ Array available from Amersham Biosciences. Another array that is useful is one that is manufactured using inkjet printing methods such as SurePrint™ Technology available from Agilent Technologies.

Other useful array substrates include those that are used in nucleic acid sequencing applications. For example, arrays that are used to create attached amplicons of genomic fragments (often referred to as ‘clusters’) can be particularly useful. Examples of substrates that can be modified for use herein include those described in Bentley et al., Nature 456:53-59 (2008), PCT Pub. Nos. WO 91/06678; WO 04/018497 or WO 07/123744; U.S. Pat. Nos. 7,057,026; 7,211,414; 7,315,019; 7,329,492 or 7,405,281; or U.S. Pat. App. Pub. No. 2008/0108082, each of which is incorporated herein by reference.

An array can have features that are separated by less than 100 μm, 50 μm, 10 μm, 5 μm, 1 μm, or 0.5 μm. In particular embodiments, features of an array can each have an area that is larger than about 100 nm², 250 nm², 500 nm², 1 μm², 2.5 μm², 5 μm²10 μm², 100 μm², or 500 μm². Alternatively or additionally, features of an array can each have an area that is smaller than about 1 mm², 500 μm², 100 μm², 25 μm², 10 μm², 5 μm², 1 μm², 500 nm², or 100 nm². Indeed, features can be separated from each other by a distance that is in a range between an upper and lower limit selected from those exemplified above. An array can have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher. An embodiment of the methods set forth herein can be used to image an array at a resolution sufficient to distinguish features at the above densities or feature separations.

An array or other multiplex format can be used to sequence at least 10, 100, 1×10³, 1×10⁴, 1×10⁵, 1×10⁶ or more different nucleic acids. Alternatively or additionally, the number of different nucleic acids that are sequenced in an array or other multiplex format can be at most 1×10⁶, 1×10⁵, 1×10⁴, 1×10³, 100 or 10. Each of the different nucleic acids can be present as a single molecule or as a member of an ensemble (e.g. the ensemble can be a feature on an array). Each of the nucleic acids in a multiplex format can produce a trace that is processed as set forth herein. Optionally, multiple traces can be produced from each nucleic acid. For example, four color sequencing methods can be used such that each nucleotide type in a sequence produces one of four different colored signals and such that each different nucleic acid produces four traces. The different signals need not be distinguished by color and can instead be distinguished based on other signal characteristics set forth herein or known in the art.

A particularly useful vessel for use in a method of the present disclosure is a flow cell. Any of a variety of flow cells can be used including, for example, those that include at least one channel and openings at either end of the channel. The openings can be connected to fluidic components to allow reagents to flow through the channel. The flow cell is generally configured to allow detection of analytes within the channel, for example, in the lumen of the channel or on the inner surface of a wall that forms the channel. In some embodiments, the flow cell can include a plurality of channels each having openings at their ends.

Several embodiments utilize optical detection of analytes in a flow cell. Accordingly, a flow cell can include one or more channels each having at least one transparent window. In particular embodiments, the window can be transparent to radiation in a particular spectral range including, but not limited to x-ray, ultraviolet (UV), visible (VIS), infrared (IR), microwave and/or radiowave radiation. In some cases, analytes are attached to an inner surface of the window(s). Alternatively or additionally, one or more windows can provide a view to an internal substrate to which analytes are attached. Exemplary flow cells and physical features of flow cells that can be useful in a method or apparatus set forth herein are described, for example, in US Pat. App. Pub. No. 2010/0111768 A1, WO 05/065814 or US Pat. App. Pub. No. 2012/0270305 A1, each of which is incorporated herein by reference in its entirety.

Particular embodiments of the present methods will capture a collection of signals from an array at relatively high resolution. For example, a detection system can be used to resolve features (e.g. nucleic acid features) on a surface that are separated by less than 100 μm, 50 μm, 10 μm, 5 μm, 1 μm, or 0.5 μm. The detection system can be configured to resolve features having an area on a surface that is smaller than about 1 mm², 500 μm², 100 μm², 25 μm², 10 μm², 5 μm², 1 μm², 500 nm², or 100 nm².

In particular embodiments, an apparatus or method can employ optical sub-systems or components used in nucleic acid sequencing systems. Several such detection apparatus are configured for optical detection, for example, detection of luminescent or fluorescent signals. Examples of detection apparatus and components thereof that can be used to detect a vessel herein are described, for example, in US Pat. App. Pub. No. 2010/0111768 A1 or U.S. Pat. Nos. 7,329,860; 8,951,781or 9,193,996, each of which is incorporated herein by reference. Other detection apparatus include those commercialized for nucleic acid sequencing such as those provided by Illumina™, Inc. (e.g. HiSeq™, MiSeq™, NextSeq™, or NovaSeq™ systems), Life Technologies™ (e.g. ABI PRISM™, or SOLID™ systems), Pacific Biosciences (e.g. systems using SMRT™ Technology such as the Sequel™ or RS II™ systems), or Qiagen (e.g. Genereader™ system). Other useful detectors are described in U.S. Pat. Nos. 5,888,737; 6,175,002; 5,695,934; 6,140,489; or 5,863,722; or US Pat. Pub. Nos. 2007/007991 A1, 2009/0247414 A1, or 2010/0111768; or WO2007/123744, each of which is incorporated herein by reference in its entirety.

A detection apparatus that is used in a method or apparatus set forth herein need not be capable of optical detection. For example, the detector can be an electronic detector used for detection of protons or pyrophosphate (see, for example, US Pat. App. Pub. Nos. 2009/0026082 A1; 2009/0127589 A1; 2010/0137143 A1; or 2010/0282617 A1, each of which is incorporated herein by reference in its entirety, or the Ion Torrent™ systems commercially available from ThermoFisher, Waltham, Mass.) or as used in detection of nanopores such as those commercialized by Oxford Nanopore™ Oxford UK (e.g. MinION™ or PromethION™ systems) or set forth in U.S. Pat. No. 7,001,792; Soni & Meller, Clin. Chem. 53, 1996-2001 (2007); Healy, Nanomed. 2, 459-481 (2007); or Cockroft, et al. J. Am. Chem. Soc. 130, 818-820 (2008), each of which is incorporated herein by reference.

Particular embodiments utilize processes acting under control of instructions and/or data stored in or transferred through one or more computer systems. Certain embodiments also relate to an apparatus for performing these operations. This apparatus may be specially designed and/or constructed for the required purposes, for example, sequencing nucleic acids, or it may be a general-purpose computer selectively configured by one or more computer programs and/or data structures stored in or otherwise made available to the computer. The processes presented herein are not inherently related to any particular computer or other apparatus. In particular, various general-purpose machines may be used with programs written in accordance with the present disclosure, or it may be more convenient to construct a more specialized apparatus to perform the required method steps.

Some embodiments relate to computer readable media or computer program products that include program instructions and/or data for performing various computer-implemented operations associated with at least the following tasks: (1) obtaining signal data from a nucleic acid sequencing procedure (e.g. image data acquired from an array of nucleic acid features subjected to a sequencing procedure); (2) extracting signals from individual nucleic acid features in an array or from other individual nucleic acids in a multiplex nucleic acid sample; (3) comparing multiple signal traces for different nucleotide types at an individual feature of an array; (4) applying a baseline adjustment, smoothing algorithm and/or other correction algorithm to individual extracted signal traces; (5) applying a linear interpolation function to a extracted signal trace for each nucleotide at a particular feature of an array. This disclosure also provides computational apparatus executing instructions to perform any or all of these tasks. It also provides computational apparatus including computer readable media encoded with instructions for performing such tasks.

A particularly useful computer system can include: one or more processors; one or more computer-readable storage media having stored thereon signal data from a nucleic acid sequencing procedure carried out on an array of nucleic acid features; and one or more computer-readable storage media storing program code that, when executed by the one or more processors, causes the computer system to implement a method for determining nucleic acid sequences, the program code including: (a) code for extracting signals from each nucleic acid feature to produce multiple extracted signal traces, wherein each extracted signal trace correlates signal characteristics with sequencing cycle for a particular nucleotide type at a particular nucleic acid feature; (b) code for comparing the extracted signal traces for different nucleotide types at each of the features, thereby distinguishing an extracted signal having a characteristic of a candidate base call from extracted background signals for each cycle at each feature; (c) code for applying a baseline adjustment to each extracted signal trace based on the extracted background signals, thereby obtaining a adjusted signal trace for each nucleotide at each feature; and (d) code for comparing the adjusted signal traces for different nucleotide types at each of the features, thereby distinguishing adjusted signals having characteristics of a base call from adjusted background signals for each cycle at each feature, whereby nucleic acid sequences are determined from the sequence of the base calls at each of the features.

A computer system of the present disclosure can be configured to communicate with an apparatus for sequencing nucleic acids. For example, the computer system can be an integral component of a nucleic acid sequencing apparatus. Optionally, a sequencing apparatus includes components and reagents for performing one or more steps set forth herein including, but not limited to, fluidic steps for delivering sequencing reagents to an array of nucleic acids, detection steps for examining and acquiring signals from sequencing reactions, and signal processing hardware for performing baseline correction and/or base calling. Alternatively, the computer system can be a separate component of a distributed system. A computer system that is used to analyze signal data can be in communication with a sequencing apparatus, for example, via wired or wireless communication.

A nucleic acid sequencing apparatus of the present disclosure can include a vessel or solid support for carrying out a nucleic acid sequencing method. For example, the apparatus can include an array, flow cell, multi-well plate or other convenient vessel for sequencing nucleic acids. The vessel or solid support can be removable, thereby allowing it to be placed into or removed from the apparatus. As such, a sequencing apparatus can be configured to sequentially process a plurality of vessels or solid supports. The system can include a fluidic system having reservoirs for containing one or more of the reagents set forth herein (e.g. polymerase, primer, template nucleic acid, nucleotide(s) for ternary complex formation, nucleotides for primer extension, deblocking reagents or mixtures of such components). The fluidic system can be configured to deliver reagents to a vessel, for example, via channels or droplet transfer apparatus (e.g. electrowetting apparatus).

Optionally, signal processing methods set forth herein are programmed in a computer processing unit (CPU). In particular embodiments, a CPU can be used to determine, from the signals, the identity of the nucleotide that is present at a particular location in a template nucleic acid. In some cases, the CPU will identify a sequence of nucleotides for the template from the signals that are detected. In particular embodiments, the CPU is programmed to correct signal traces. An exemplary algorithm that can be run on a CPU (or other processor hardware) of a system is diagramed in FIG. 1, and exemplary code is provided in Appendix 1.

A useful CPU can include one or more of a personal computer system, server computer system, thin client, thick client, hand-held or laptop device, multiprocessor system, microprocessor-based system, set top box, programmable consumer electronic, network PC, minicomputer system, mainframe computer system, smart phone, and distributed cloud computing environments that include any of the above systems or devices, and the like. The CPU can include one or more processors or processing units, a memory architecture that may include RAM and non-volatile memory. The memory architecture may further include removable/non-removable, volatile/non-volatile computer system storage media. Particularly useful are tangible computer-readable media. Examples of tangible computer-readable media suitable for use with computer program products and computational apparatus of this invention include, but are not limited to, magnetic media such as hard disks, floppy disks, and magnetic tape; optical media such as CD-ROM disks; magneto-optical media; semiconductor memory devices (e.g., flash memory), and hardware devices that are specially configured to store and perform program instructions, such as read-only memory devices (ROM) and random access memory (RAM) and sometimes application-specific integrated circuits (ASICs), programmable logic devices (PLDs) and signal transmission media for delivering computer-readable instructions, such as local area networks, wide area networks, and the Internet. The data and program instructions provided herein may also be embodied on a carrier wave or other transport medium (e.g., optical lines, electrical lines, and/or airwaves).

Further, the memory architecture may include one or more readers for reading from and writing to tangible computer-readable media, or for reading from and writing to a non-removable, non-volatile magnetic media. A CPU may also include a variety of computer system readable media. Such media may be any available media that is accessible by a cloud computing environment, such as volatile and non-volatile media, and removable and non-removable media.

The memory architecture may include at least one program product having at least one program module implemented as executable instructions that are configured to carry out one or more steps of a method set forth herein. For example, executable instructions may include an operating system, one or more application programs, other program modules, and program data. Generally, program modules may include routines, programs, objects, components, logic, data structures, and so on, that perform particular tasks set forth herein. Signal data can be captured and stored in the memory architecture of a computer system. The signal data that is stored in memory can be raw signal data or the data can be processed, for example, to create a signal trace such as a signal trace having a format exemplified herein.

The components of a CPU may be coupled by an internal bus that may be implemented as one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

A CPU can optionally communicate with one or more external devices such as a keyboard, a pointing device (e.g. a mouse), a display, such as a graphical user interface (GUI), or other device that facilitates interaction of a use with the nucleic acid detection system. Examples of displays suitable for interfacing with a user in accordance with the present disclosure include but are not limited to cathode ray tube displays, liquid crystal displays, plasma displays, touch screen displays, video projection displays, light-emitting diode and organic light-emitting diode displays, surface-conduction electron-emitter displays and the like. Examples of printers include toner-based printers, liquid inkjet printers, solid ink printers, dye-sublimation printers as well as inkless printers such as thermal printers. Printing may be to a tangible medium such as paper or transparencies.

Similarly, the CPU can communicate with other devices (e.g., via network card, modem, etc.). Such communication can occur via I/O interfaces. Still yet, a CPU of a system herein may communicate with one or more networks such as a local area network (LAN), a general wide area network (WAN), and/or a public network (e.g., the Internet) via a suitable network adapter.

### EXAMPLE I

### Baseline Correction

This example demonstrates correction of extracted signal data from a Sequencing By Binding™ (SBB™) platform to improve the quality of base calls and reduce the percent error of base calls relative to the known reference sequence. In SBB techniques that utilize luminescence detection of labeled nucleotides, signals are apparent from both the correct nucleotide (the ‘on’ signal) and the three incorrect nucleotides (the ‘off’ signals), where the assumption is that the highest intensity signal is the ‘on’ signal. However, if there is a trend where the ‘off’ signal intensities increase by different amounts for each nucleotide over multiple SBB™ cycles, then an incorrect base call may be made due to a nucleotide with an ‘off’ baseline intensity that is higher than a nucleotide that is ‘on’ but has a lower baseline. The remedy described here is intended to determine the ‘off’ signal intensities for each nucleotide and each array feature examined in each of the SBB™ cycles, and correct the baseline such that the ‘off’ signal intensity has a value of zero on average.

The correction is implemented in Python with the source code shown in Appendix 1. A diagram of the algorithm is shown in FIG. 1. The pseudo code for the Python implementation is as follows:
Iterate over the features in the sequencing run
   Iterate over the SBB sequencing cycles
      In each cycle, sort the four intensities and store the smallest three in a vector with the cycle label for each nucleotide
Iterate over each exam
   Smooth the vector by averaging points within a window
   Since some cycles may still not have a value if a nucleotide had the maximum intensity for the whole window, linearly interpolate the smooth window, but do not extrapolate
   Fill out the beginning and end cycles of the window with the first and last smooth window value, respectively
   Subtract the smoothed, interpolated ‘off’ intensity values from the intensity for that nucleotide in each cycle

A demonstration of the correction technique and its beneficial impact on base calling is provided by FIG. 2. FIG. 2A shows a plot of raw signal traces (of signal intensity vs. cycle) for A, C, T and G signals that have been extracted from a single nucleic acid cluster having been subjected to a Sequencing By Binding™ procedure. Also shown in the figure is a reference sequence (top line) and base calls derived from the raw signal data (second line). Eighteen miscalls (all G's) are indicated by a subscript offset to the second line. In all eighteen cases, miscall results from an elevation in baseline for the G signal trace where a peak for the correct base has an intensity below the elevated baseline for the G signal. FIG. 2B shows a plot of adjusted signal traces (signal intensities vs. cycle) for the A, C, T and G signals after a single iteration of baseline correction for the raw signal traces shown in FIG. 2A. Again, the reference sequence is shown on the top line and the base calls are shown in the second line. After baseline correction one miscall remained. Two more iterations of baseline correction were carried out on the signal traces shown in FIG. 2B and the results are shown in FIG. 2C. As indicated by the reference sequence and aligned base calls, three iterations of the baseline correction algorithm removed all miscalls from the sequence that would have been called from the raw signal traces.

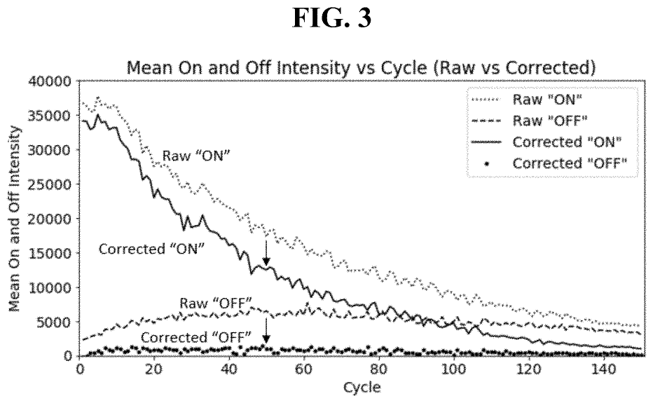

FIGS. 3 through 5 further demonstrate the efficacy of this correction in lowering error rate by comparing results of base calling using the same data set with and without the correction.

As shown in FIG. 3, without the correction the ‘off’ signal intensities are near 5000 counts in the beginning of the run. After applying the correction, the ‘off’ signal intensities are zero on average and the ‘on’ signal intensities are shifted down by the baseline subtraction.

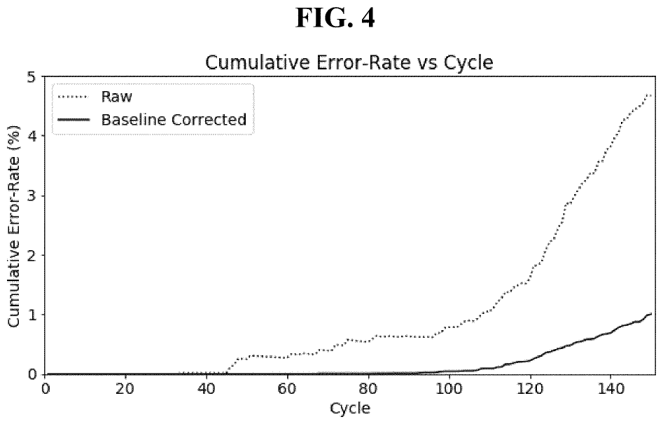

A comparison of the plot of cumulative error versus cycle before and after applying the correction (FIG. 4) shows that the correction is effective in reducing sequencing errors. In this case, the cumulative error relative to the ten reference sequences at 100 cycles was reduced from about 0.9% to about 0.1% by the baseline correction algorithm.

When the error was broken down by reference sequence, it was apparent that reference sequence 6 was driving a significant portion of error in cycles 60-80. The baseline correction removed most of that error. Without the correction, the ‘off’ signal intensity in the C channel was higher than the other three nucleotides. At cycle 60, the ‘off’ C signal intensity was near the magnitude of the ‘on’ signal intensity which resulted in incorrectly calling bases as C. After baseline correction, the differential rise in ‘off’ signal intensity was removed and the correct base call results.

One alternative of the baseline correction method of this example, is to fit the correction to a functional form instead of doing interpolation between smoothed data points. For example, the current shape of the ‘off’ signal baseline appears to be an exponential growth followed by an exponential decay, which could be modeled for each feature and nucleotide over SBB™ sequencing cycles.

Other sequencing technologies, such as Sequencing By Synthesis approaches, perform signal corrections also with a goal of the ‘off’ signal intensities being zero. These corrections may adjust for characteristics of the data acquisition system, such as optical crosstalk, or they may adjust for biochemical phenomena, such as phasing artifacts. The limitation of these corrections is that they assume a model for the source of ‘off’ signal increase and then determine the coefficients for that model. In the case of phasing correction, coefficients are determined to multiply the previous cycle signal intensities and the next cycle signal intensities to correct the current cycle signal intensities. While there are different coefficients for the previous and next cycle, there isn't a separate correction for individual nucleotides nor for individual features in an array of nucleic acids that is being sequenced.

The approach demonstrated in this Example is unique in that it is a correction for each feature in the array being sequenced, each type of nucleotide that is examined in the sequencing procedure, and each cycle of the sequencing procedure to effectively correct every signal being processed. The baseline correction is determined by inspecting the data and there is no need to make any assumption of a model or a functional form for the correction. However, the correction exemplified here can be implemented in combination with a model. Therefore, the method exemplified herein can correct for a wide variety of sources of elevated ‘off’ signal baseline.

Throughout this application various publications, patents and/or patent applications have been referenced. The disclosures of these documents in their entireties are hereby incorporated by reference in this application.

A number of embodiments have been described. Nevertheless, it will be understood that various modifications may be made. Accordingly, other embodiments are within the scope of the following claims.

```
APPENDIX 1
import numpy as np
import copy
from scipy.interpolate import interp1d
def subtract_baseline_off_intensities (traces, number_of_cycles, exams_per_cycle, exam_is_n, cycle_window=9) :
  if cycle_window % 2 != 1 :
    raise ValueError, “Off intensity baseline subtraction cycle window size of {0} is not
odd.”.format (cycle_window)
  baseline_subtracted_traces = copy.deepcopy (traces)
  baseline_off_traces = np.zeros (traces.shape)
  if exams_per_cycle != 4 :
    return baseline_subtracted_traces, baseline_off_traces
  number_of_spots = traces.shape [0]
  for spot in range (number_of_spots) :
    baseline = np.full ( (exams_per_cycle, number_of_cycles), np.nan)
    for cycle in range (number_of_cycles) :
      # Get a per exam baseline vector of the non-N calls excluding the brightest intensity per cycle
      if not np.any (exam_is_n [spot, cycle*exams_per_cycle: (cycle+1) *exams_per_cycle] ) :
        intensity_vector = traces [spot,cycle*exams_per_cycle: (cycle+1) *exams_per_cycle]
        sorted_indices = np.argsort (intensity_vector)
        for sorted_index in range (exams_ per_cycle − 1) :
          exam = sorted_indices [sorted_index]
          baseline [exam, cycle] = intensity_vector [exam]
    half_window = cycle_window / 2
    x_values = np.arange (0.0, number_of_cycles, 1.0)
    for exam in range (exams_per_cycle):
      # smooth the baseline vector using the mean over a window, excluding cycles without data
      smooth_baseline = np.full (number_of_cycles, np.nan)
      for cycle in range (half_window, number_of_cycles − half_window) :
        baseline_window = baseline [exam, cycle−half_window:cycle+half_window+1]
        if np.isfinite (baseline_window) .any ( ) :
          smooth_baseline [cycle] = np.nanmean (baseline_window)
      # there may be empty entries due to N, homopolymers, and the beginning and end of read
      # linearly interpolate the rest of the baseline from the smooth baseline
      non_nan_indices = np.isfinite (smooth_baseline)
      interp_x = x_values [non_nan_indices]
      interp_y = smooth_baseline [non_nan_indices]
      if len (interp_x) > 1:
        interp_func = interp1d (interp_x, interp_y, kind=‘linear’, assume_sorted=True)
        interp_baseline = interp_func (np.arange (interp_x [0], interp_x [−1]+1.0,1.0) )
        min_index = np.where (non_nan_indices) [0] [0]
        max_index = np.where (non_nan_indices) [0] [−1]
        smooth_baseline [min_index:max_index+1] = interp_baseline
        smooth_baseline [ :min_index] = smooth_baseline [min_index]
        smooth_baseline [max_index+1: ] = smooth_baseline [max_index]
      else:
        smooth_baseline [ : ] = 0.0
      # subtract the baseline from non-N cycles
      for cycle in range (number_of_cycles) :
        if not exam_is_n [spot, cycle*exams_per_cycle+exam] :
          baseline_subtracted_traces [spot, cycle*exams_per_cycle+exam] −= smooth_baseline [cycle]
      baseline_off_traces [spot, exam:number_of_cycles*exams_per_cycle:exams_per_cycle] = smooth_baseline
  return baseline_subtracted_traces, baseline_off_traces
```

What is claimed is:

1. A method of determining nucleic acid sequences, comprising:
(a) obtaining signal data from a nucleic acid sequencing procedure carried out on an array of nucleic acid features;
(b) extracting signals from each nucleic acid feature to produce multiple extracted signal traces, wherein each extracted signal trace correlates signal characteristics with sequencing cycle for a particular nucleotide type at a particular nucleic acid feature;
(c) comparing the extracted signal traces for different nucleotide types at each of the features, thereby distinguishing an extracted signal having a characteristic of a candidate base call from extracted background signals for each cycle at each feature;
(d) applying a baseline adjustment to each extracted signal trace based on the extracted background signals, thereby obtaining an adjusted signal trace for each nucleotide at each feature;
(e) comparing the adjusted signal traces for different nucleotide types at each of the features, thereby distinguishing adjusted signals having characteristics of a base call from adjusted background signals for each cycle at each feature, whereby nucleic acid sequences are determined from the sequence of the base calls at each of the features.

2. The method of claim 1, wherein the signals comprise luminescent signals and step (a) comprises obtaining luminescent images of the array.

3. The method of claim 2, wherein different nucleotide types produce luminescent signals at different wavelengths.

4. The method of claim 1, wherein the signal characteristic comprises luminescence intensity.

5. The method of claim 4, wherein the extracted signal having the highest luminescence intensity for a particular cycle and particular feature is identified as the candidate base call for the particular cycle and the particular feature,
wherein the other extracted signals for the particular cycle and the particular feature are identified as background signals
wherein the adjusted signal having the highest luminescence intensity for a particular cycle and particular feature is identified as the base call for the particular cycle and the particular feature, and
wherein the other adjusted signals for the particular cycle and the particular feature are identified as background signals.

6. The method of claim 4, wherein the extracted signal having the lowest luminescence intensity for a particular cycle and particular feature is identified as the candidate base call for the particular cycle and the particular feature,
wherein the other extracted signals for the particular cycle and the particular feature are identified as background signals
wherein the adjusted signal having the lowest luminescence intensity for a particular cycle and particular feature is identified as the base call for the particular cycle and the particular feature, and
wherein the other adjusted signals for the particular cycle and the particular feature are identified as background signals.

7. The method claim 1, wherein each feature produces the signal for the candidate base call and three background signals indicative of three other types of nucleotides.

8. The method of claim 1, wherein the signal characteristic comprises a difference in signal intensities between a first nucleotide type and at least one other nucleotide type for a particular nucleic acid feature at a particular cycle.

9. The method of claim 1, wherein the extracted signal that is characteristic of the candidate base call has signal intensity that is greater than signal intensities for the extracted background signal, and wherein the adjusted signal that is characteristic of the candidate base call has signal intensity that is greater than signal intensities for the adjusted background signals.

10. The method of claim 1, wherein the extracted signal that is characteristic of the candidate base call has signal intensity that is lower than signal intensities for the extracted background signal, and wherein the adjusted signal that is characteristic of the candidate base call has signal intensity that is lower than signal intensities for the adjusted background signals.

11. The method of claim 1, wherein the baseline adjustment comprises a smoothing function.

12. The method of claim 11, wherein the adjusting of step (d) further comprises applying an interpolation function to the extracted signal trace for each nucleotide at each feature.

13. The method of claim 1, wherein step (d) comprises:
(i) applying a baseline adjustment to each extracted signal trace based on the extracted background signals, thereby obtaining a adjusted signal trace for each nucleotide at each feature,
(ii) comparing the adjusted signal traces for different nucleotide types at each of the features, thereby distinguishing an adjusted signal having a characteristic of a candidate base call from adjusted background signals for each cycle at each feature, and
(iii) applying a baseline adjustment to each adjusted signal trace based on the adjusted background signals, thereby obtaining a series of iteratively adjusted signals for each nucleotide at each feature.

14. The method of claim 11, wherein step (d) further comprises repeating steps (d)(i) through (d)(iii) using the iteratively adjusted series of signals in place of the adjusted series of signals.

15. The method of claim 1, wherein step (a) comprises:
(i) contacting the array of nucleic acid features with reagents for forming ternary complexes, wherein the reagents comprise a polymerase and nucleotide cognates for at least three different base types suspected of being present in the nucleic acids,
(ii) acquiring signals from the features while precluding polymerase catalyzed extension of the nucleic acids at the features,
(iii) after step (a)(ii), extending the nucleic acids to produce extended nucleic acids at the features, and
(iv) repeating steps (a)(i) through (iii) for the extended nucleic acids at the features.

16. The method of claim 15, wherein the nucleotide cognates for at least three different base types are attached to exogenous labels that produce the signals.

17. The method of claim 15, wherein the nucleic acids are extended by addition of a reversibly terminated nucleotide to each nucleic acid at the features in step (a)(iii).

18. The method of claim 15, wherein the polymerase catalyzed extension is precluded by the presence of a reversible terminator on the nucleic acids at the features.

19. The method of claim 18, further comprising deblocking and extending the nucleic acids at the features after step (a)(ii) and before step (a)(iii).

20. The method of claim 15, wherein the extracted signal for the candidate base call is produced by ternary complex comprising the next correct nucleotide.

21. The method of claim 1, wherein the array comprises at least 1×10³ features that produce the signal data, whereby 1×10³ nucleic acid sequences are determined from 1×10³ series of base calls.

22. A computer system, comprising:
one or more processors;
one or more computer-readable storage media having stored thereon signal data from a nucleic acid sequencing procedure carried out on an array of nucleic acid features; and
one or more computer-readable storage media storing program code that, when executed by the one or more processors, causes the computer system to implement a method for determining nucleic acid sequences, the program code comprising:
(a) code for extracting signals from each nucleic acid feature to produce multiple extracted signal traces, wherein each extracted signal trace correlates signal characteristics with sequencing cycle for a particular nucleotide type at a particular nucleic acid feature;
(b) code for comparing the extracted signal traces for different nucleotide types at each of the features, thereby distinguishing an extracted signal having a characteristic of a candidate base call from extracted background signals for each cycle at each feature;
(c) code for applying a baseline adjustment to each extracted signal trace based on the extracted background signals, thereby obtaining a adjusted signal trace for each nucleotide at each feature; and
(d) code for comparing the adjusted signal traces for different nucleotide types at each of the features, thereby distinguishing adjusted signals having characteristics of a base call from adjusted background signals for each cycle at each feature, whereby nucleic acid sequences are determined from the sequence of the base calls at each of the features.


SEQUENCE LISTING (3 sequences)

SEQ ID NO: 1 - DNA, 70 bp, Artificial Sequence
  Description of Artificial Sequence Synthetic oligonucleotide
ctttggggagggcgggtggaaggacggatggcaggccggctggtaggtcggttgagagcgagtgaaagaa

SEQ ID NO: 2 - DNA, 70 bp, Artificial Sequence
  Description of Artificial Sequence Synthetic oligonucleotide
ctttggggagggcggggggaaggacggggggggggccggggggggggggggtgggggggggggggaagaa

SEQ ID NO: 3 - DNA, 70 bp, Artificial Sequence
  Description of Artificial Sequence Synthetic oligonucleotide
ctttggggagggcgggtggaaggacggatggcaggccggctggtagggcggttgagagcgagtgaaagaa